(19) United States
(12) Patent Application Publication
(10) Pub. No.: US 2017/0063107 A1
(43) Pub. Date: Mar. 2, 2017

(54) METHOD AND ELECTRONIC DEVICE FOR OBTAINING BIO SIGNALS

(71) Applicant: Samsung Electronics Co., Ltd., Gyeonggi-do (KR)

(72) Inventors: Seungho LEE, Seoul (KR); Myeongbo KIM, Gyeonggi-do (KR); Youngkyun BAN, Gyeonggi-do (KR); Sunlyeong HWANG, Gyeonggi-do (KR)

(73) Assignee: Samsung Electronics Co., Ltd.

(21) Appl. No.: 15/250,343

(22) Filed: Aug. 29, 2016

(30) Foreign Application Priority Data
Aug. 27, 2015 (KR) 10-2015-0120958

(51) Int. Cl. H02J 7/00; A61B 5/00

(52) U.S. Cl. CPC H02J 7/007; H02J 7/0068; A61B 5/0002; A61B 5/6801; H04B 1/385

(57) ABSTRACT

A wearable electronic device is provided that includes a housing, a battery disposed in the housing, and a coupling member, connected to the housing, for detachably coupling the electronic device to a part of a user's body. The wearable electronic device also includes a conductive member exposed on a side of the housing or on a side of the coupling member and electrically connected to the battery. The conductive member includes one or more contacts. The wearable electronic device further includes at least one sensor electrically connected to the conductive member, and a circuit electrically connected to the battery, the conductive member, and the at least one sensor. The circuit monitors a voltage and an amount of current received via the one or more contacts, and charges the battery with the current or operates the at least one sensor, based on at least one of the voltage and the amount of current.

### PRIORITY

This application claims priority under 35 U.S.C. §119(a) to Korean Patent Application No. 10-2015-0120958, filed in the Korean Intellectual Property Office on Aug. 27, 2015, the disclosure of which is incorporated herein by reference.

### BACKGROUND

1. Field of the Disclosure

The present disclosure relates to a method and an apparatus for obtaining bio signals.

2. Description of Related Art

An electronic device performs functions corresponding to programs installed thereon. Examples of the electronic device are home appliances, electronic-organizers, portable multimedia players, mobile communication terminals, tablet PCs, audio/video systems, desktop computers, laptop computers, vehicle navigation systems, etc. Electronic devices are capable of outputting the stored information in audio or video format. With the development of technologies related to integration, high speed communication, and wireless communication transferring a large amount of data, electronic devices have been equipped with various functions.

Electronic devices are equipped with various types of sensors and provide corresponding services by using sensed information.

Electronic devices have been developed in a wearable form to be worn on the human body (hereafter referred to as a wearable device). For example, a wearable device may include a contact configured to contact the human skin in order to measure biometric information by a bio sensor.

When a wearable device is designed to include an additional structure (e.g., a contact revealed outside from the case, or housing) in order to use the bio sensor, this may cause the product design to be restricted. Furthermore, manufactures may have difficulty in designing the device to include an additional structure or part.

### SUMMARY

The present disclosure has been made to address at least the above problems and/or disadvantages, and to provide at least the advantages described below. Accordingly, an aspect of the present disclosure provides an electronic device with a contact (e.g., a contact of a bio sensor) capable of being used for a number of purposes.

In accordance with an aspect of the present disclosure, a wearable electronic device is provided that includes a housing, a battery disposed in the housing, and a coupling member, connected to the housing, for detachably coupling the electronic device to a part of a user's body. The wearable electronic device also includes a conductive member exposed on a side of the housing or on a side of the coupling member and electrically connected to the battery. The conductive member includes one or more contacts. The wearable electronic device further includes at least one sensor electrically connected to the conductive member, and a circuit electrically connected to the battery, the conductive member, and the at least one sensor. The circuit monitors a voltage and an amount of current received via the one or more contacts, and charges the battery with the current or operates the at least one sensor, based on at least one of the voltage and the amount of current.

In accordance with another aspect of the present disclosure, a method is provided for obtaining a bio signal by an electronic device. A voltage and an amount of current received via the one or more contacts are monitored. The battery with the current is charged. At least one bio sensor is operated based on at least one of the voltage and the amount of current.

### BRIEF DESCRIPTION OF THE DRAWINGS

The above and other aspects, features, and advantages of the present disclosure will be more apparent from the following detailed description when taken in conjunction with the accompanying drawings, in which:

FIG. 1 is a diagram illustrating an electronic device in a network environment, according to an embodiment of the present disclosure;

FIG. 2 is a block diagram illustrating an electronic device, according to an embodiment of the present disclosure;

FIG. 3 is a block diagram illustrating a programming module, according to an embodiment of the present disclosure;

FIG. 4 is a diagram illustrating the appearance of an electronic device, according to an embodiment of the present disclosure;

FIG. 5 is a block diagram illustrating an electronic device, according to an embodiment of the present disclosure;

FIG. 6 is a circuit diagram illustrating an electronic device, according to an embodiment of the present disclosure;

FIG. 7 is a flow diagram illustrating operations of an electronic device, according to an embodiment of the present disclosure;

FIG. 8 is a graph showing a battery charging method, according to an embodiment of the present disclosure;

FIG. 9 is a flow diagram illustrating operations of an electronic device, according to an embodiment of the present disclosure;

FIG. 10 is a circuit diagram illustrating an electronic device, according to an embodiment of the present disclosure; and

FIG. 11 is a circuit diagram illustrating an electronic device, according to an embodiment of the present disclosure.

### DETAILED DESCRIPTION

Embodiments of the present disclosure are described in detail with reference to the accompanying drawings. The same or similar components may be designated by the same or similar reference numerals although they are illustrated in different drawings. Detailed descriptions of constructions or processes known in the art may be omitted to avoid obscuring the subject matter of the present disclosure.

The terms and words used in the following description and claims are not limited to their dictionary meanings, but are merely used to enable a clear and consistent understanding of the present disclosure. Accordingly, it should be apparent to those skilled in the art that the following description of various embodiments of the present disclosure is provided for illustration purposes only, and not for the purpose of limiting the present disclosure as defined by the appended claims and their equivalents.

It is to be understood that the singular forms “a”, “an”, and “the”, include plural referents unless the context clearly dictates otherwise. Thus, for example, reference to “a component surface” includes reference to one or more of such surfaces.

Expressions such as “include” and “may include”, as used herein, denote the presence of the disclosed functions, operations, and constituent elements, and do not limit one or more additional functions, operations, and constituent elements. Herein, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

Herein, the expression “and/or” includes any and all combinations of the associated listed words. For example, the expression “A and/or B” may include A, may include B, or may include both A and B.

Herein, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first user device and a second user device indicate different user devices, although both are user devices. Additionally, a first element may be referred to as a second element, and similarly, a second element may also be referred to as a first element, without departing from the scope of the present disclosure.

When a component is referred to as being “connected to” or “accessed by” another component, it should be understood that not only is the component connected or accessed to the other component, but also another component may exist between the component and the other component. When a component is referred to as being “directly connected to” or “directly accessed by” another component, it should be understood that there is no component therebetween.

The terms used herein are used to describe specific embodiments, and are not intended to limit the present disclosure.

Unless otherwise defined, all terms including technical and/or scientific terms used herein have the same meaning as commonly understood by one of ordinary skill in the art to which the present disclosure pertains. In addition, unless otherwise defined, all terms defined in generally used dictionaries may not be overly interpreted.

The electronic device corresponds to at least one of a smartphone, a tablet personal computer (PC), a mobile phone, a video phone, an e-book reader, a desktop PC, a laptop PC, a netbook computer, a personal digital assistant (PDA), a portable multimedia player (PMP), a digital audio player (e.g., Moving Picture Experts Group phase 1 or phase 2 (MPEG-1 or MPEG-2) audio layer 3 (MP3) player), a mobile medical device, a camera, or a wearable device. Examples of the wearable device include a head-mounted-device (HMD) (e.g., electronic eyeglasses), electronic clothing, an electronic bracelet, an electronic necklace, an appcessory, an electronic tattoo, a smart watch, and the like.

The electronic device, according to embodiments of the present disclosure, may also be a smart home appliance. Examples of a smart home appliance include a television (TV), a digital versatile disc (DVD) player, an audio system, a refrigerator, an air-conditioner, a cleaning device, an oven, a microwave oven, a washing machine, an air cleaner, a set-top box, a TV box, a game console, an electronic dictionary, an electronic key, a camcorder, an electronic album, or the like.

The electronic device, according to embodiments of the present disclosure, may also include medical devices (e.g., magnetic resonance angiography (MRA), magnetic resonance imaging (MRI), computed tomography (CT), a scanning machine, an ultrasonic scanning device, and the like), a navigation device, a global positioning system (GPS) receiver, an event data recorder (EDR), a flight data recorder (FDR), a vehicle infotainment device, an electronic equipment for ships (e.g., navigation equipment, gyrocompass, and the like), avionics, a security device, a head unit for vehicles, an industrial or home robot, an automatic teller machine (ATM), a point of sales (POS) system, an Internet of Things (IoT) device, and the like.

The electronic device, according to the embodiments of the present disclosure, may also include furniture or a portion of a building/structure, an electronic board, an electronic signature receiving device, a projector, various measuring instruments (e.g., a water meter, an electric meter, a gas meter and a wave meter) and the like. The electronic device may also include a combination of the devices listed above. Additionally, the electronic device may be a flexible and/or contoured device. It should be obvious to those skilled in the art that the electronic device is not limited to the aforementioned devices.

Hereinafter, electronic devices, according to embodiments of the present disclosure, will be described in detail with reference to the accompanying drawings. In the description, the term ‘user’ may refer to a person or a device that uses or otherwise controls the electronic device, e.g., an artificial intelligence electronic device.

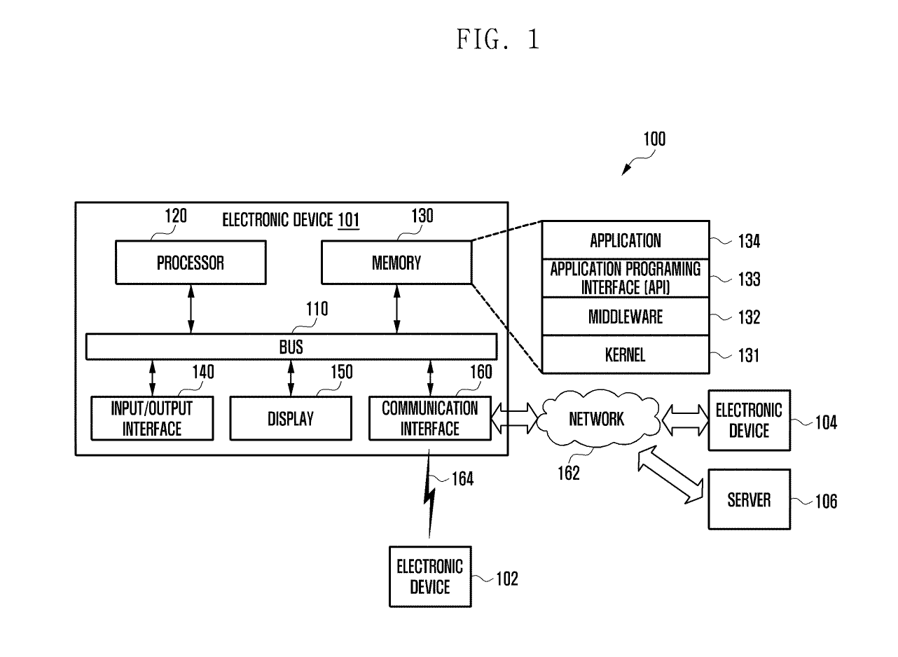

FIG. 1 is a diagram illustrating a network environment including an electronic device, according to an embodiment of the present disclosure.

Referring to FIG. 1, an electronic device 101 of a network environment 100 includes a bus 110, a processor 120, a memory 130, an input/output (I/O) interface 140, a display 150 and a communication interface 160. The bus 110 may be a communication circuit that connects the components to each other and transfers data (e.g., control messages) between the components.

The processor 120 may receive instructions from the components (e.g., the memory 130, the I/O interface 140, the display 150, and the communication interface 160) via the bus 110, decode the instructions and perform corresponding operations or data processing according to the decoded instructions.

The memory 130 may store instructions or data transferred from/created in the processor 120 or the other components (e.g., the I/O interface 140, the display 150, and the communication interface 160). The memory 130 includes programming modules, e.g., a kernel 131, a middleware 132, an application programming interface (API) 133, and an application module 134. Each of the programming modules may be software, firmware, hardware, or a combination thereof.

The kernel 131 may control or manage system resources (e.g., the bus 110, the processor 120, and the memory 130) used to execute operations or functions of the programming modules, e.g., the middleware 132, the API 133, and the application module 134. The kernel 131 may also provide an interface that can access and control/manage the components of the electronic device 101 via the middleware 132, the API 133, and the application module 134.

The middleware 132 may make it possible for the API 133 or application module 134 to perform data communication with the kernel 131. The middleware 132 may also perform control operations (e.g., scheduling and load balancing) for task requests transmitted from the application module 134 using, for example, a method for assigning the order of priority to use the system resources (e.g., the bus 110, processor 120, and memory 130) of the electronic device 101 to at least one of the applications of the application module 134.

The API 133 is an interface that allows the application module 134 to control functions of the kernel 131 or middleware 132. For example, the API 133 may include at least one interface or function (e.g., instruction) for file control, window control, character control, video process, and the like.

According to embodiments of the present disclosure, the application module 134 may include applications that are related to short message service (SMS)/multimedia messaging service (MMS), email, calendar, alarm, health care (e.g., an application for measuring blood sugar level, a workout application, and the like), and environment information (e.g., atmospheric pressure, humidity, temperature, and the like). The application module 134 may be an application related to exchanging information between the electronic device 101 and external electronic devices (e.g., a first external electronic device 102, a second external electronic device 104, and a server 106). The information exchange-related application may include a notification relay application for transmitting specific information to an external electronic device or a device management application for managing external electronic devices.

For example, the notification relay application may include a function for transmitting notification information, created by the other applications of the electronic device 101 (e.g., SMS/MMS application, email application, health care application, environment information application, and the like), to an external electronic device (e.g., the second external electronic device 104). In addition, the notification relay application may receive notification information from an external electronic device (e.g., the second external electronic device 104) and provide it to the user. The device management application can manage (e.g., install, delete, or update) part of the functions of an external electronic device (e.g., the second external electronic device 104) communicating with the electronic device 101, e.g., turning on/off the external electronic device, turning on/off part of the components of the external electronic device, adjusting the brightness or the display resolution of the display of the external electronic device, and the like, applications operated in the external electronic device, or services from the external electronic device, e.g., call service or messaging service, and the like.

According to embodiments of the present disclosure, the application module 134 may also include applications designated according to attributes (e.g., type of electronic device) of the external electronic device (e.g., the second external electronic device 104). For example, if the external electronic device is an MP3 player, the application module 134 may include an application related to music playback. If the external electronic device is a mobile medical device, the application module 134 may include an application related to health care. According to an embodiment of the present disclosure, the application module 134 may include an application designated in the electronic device 101 and applications transmitted from external electronic devices (e.g., the server 106, the second external electronic device 104, and the like).

The I/O interface 140 may receive instructions or data from the user via an I/O system (e.g., a sensor, a keyboard, or a touch screen) and transfers them to the processor 120, the memory 130, or the communication interface 160 through the bus 110. For example, the I/O interface 140 may provide data corresponding to a user's touch input to a touch screen to the processor 120. The I/O interface 140 may receive instructions or data from the processor 120, memory 130 or communication interface 160 through the bus 110, and output them to an I/O system (e.g., a speaker or a display). For example, the I/O interface 140 may output voice data processed by the processor 120 to a speaker.

The display 150 may display information (e.g., multimedia data, text data, and the like) on a screen so that the user can view it.

The communication interface 160 may communicate between the electronic device 101 and an external system (e.g., the second external electronic device 104 or the server 106). For example, the communication interface 160 may connect to a network 162 in a wireless or wired mode, and communicate with the external system. Wireless communication may include Wi-Fi, Bluetooth (BT), near field communication (NFC), GPS, or cellular communication (e.g., long term evolution (LTE), LTE-advanced (LTE-A), code division multiple access (CDMA), wideband CDMA (WCDMA), universal mobile telecommunications system (UMTS), wireless broadband (Wi-Bro), global system for mobile communications (GSM), and the like). In addition, the wireless communication may include, for example, short range communication 164. Wired communication may include universal serial bus (USB), high definition multimedia interface (HDMI), recommended standard 232 (RS-232), plain old telephone service (POTS), and the like.

In an embodiment of the present disclosure, the network 162 may be a telecommunication network. The telecommunication network may include a computer network, Internet, IoT, telephone network, and the like. The protocol for communication between the electronic device 101 and the external system, e.g., transport layer protocol, data link layer protocol, or physical layer protocol, may be supported by at least one of the application module 134, the API 133, the middleware 132, the kernel 131 and the communication interface 160.

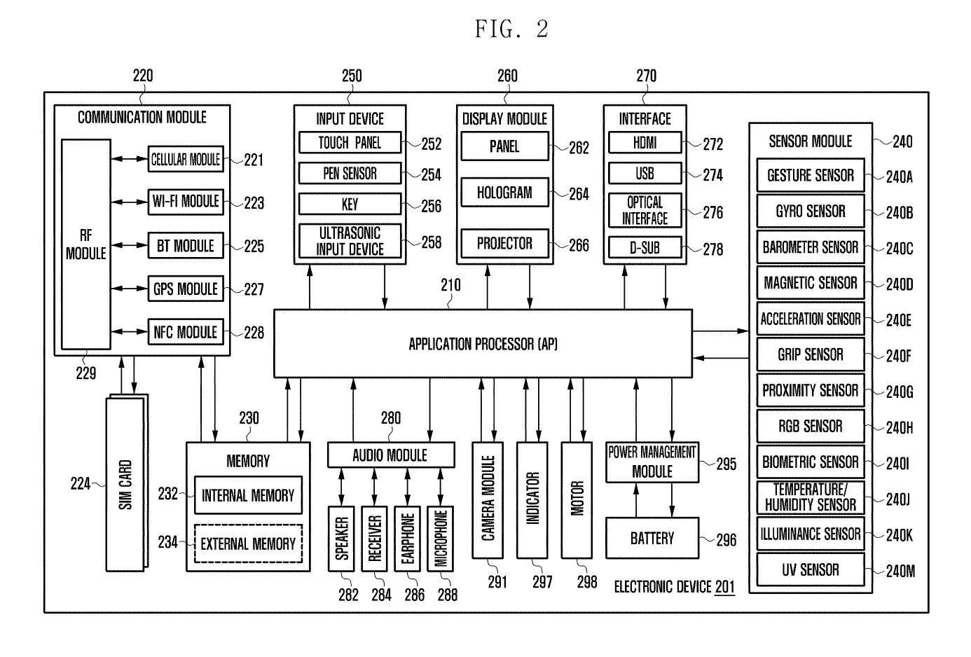

FIG. 2 is a block diagram illustrating an electronic device, according to an embodiment of the present disclosure.

Referring to FIG. 2, an electronic device 201 may be all or a part of the electronic device 101 of FIG. 1, and includes one or more processors of an application processor (AP) 210, a communication module 220, a subscriber identification module (SIM) card 224, a memory 230, a sensor module 240, an input device 250, a display module 260, an interface 270, an audio module 280, a camera module 291, a power management module 295, a battery 296, an indicator 297, and a motor 298.

The AP 210 may control a number of hardware or software components connected thereto by executing the operation system or applications, process data including multimedia data, and perform corresponding operations. The AP 210 may be implemented with a system on chip (SoC). In an embodiment of the present disclosure, the AP 210 may further include a graphics processing unit (GPU).

The communication module 220 (e.g., communication interface 160) performs communication for data transmission/reception between the other electronic devices (e.g., the first external electronic device 102, the second external electronic device 104, and the server 106) that are connected to the electronic device (e.g., the electronic device 101) via the network. In an embodiment of the present disclosure, the communication module 220 includes a cellular module 221, a Wi-Fi module 223, a BT module 225, a GPS module 227, an NFC module 228 and a radio frequency (RF) module 229.

The cellular module 221 may provide voice call, video call, SMS or Internet service, and the like, via a communication network (e.g., LTE, LTE-A, CDMA, WCDMA, UMTS, Wi-Bro, GSM, and the like). The cellular module 221 may also perform identification or authentication for electronic devices in a communication network by using their SIM (e.g., SIM card 224). In an embodiment of the present disclosure, the cellular module 221 may perform part of the functions of the AP 210. For example, the cellular module 221 may perform part of the functions for controlling multimedia.

In an embodiment of the present disclosure, the cellular module 221 may include a communication processor (CP). The cellular module 221 may be implemented with, for example, an SoC. Although the embodiment of the present disclosure shown in FIG. 2 is implemented in such a way that the cellular module 221 (e.g., CP), the power management module 295, the memory 230, and the like, are separated from the AP 210, an embodiment can be modified in such a way that the AP 210 includes at least part of the listed elements or other elements of the device 201 (e.g., the cellular module 221).

In an embodiment of the present disclosure, the AP 210 or the cellular module 221 (e.g., CP) may load instructions or data transmitted to and from at least one of a non-volatile memory or other components, on a volatile memory and then process them. The AP 210 or the cellular module 221 may also store data which is transmitted from/created in at least one of the components, in a non-volatile memory.

The Wi-Fi module 223, the BT module 225, the GPS module 227 and the NFC module 228 may include processors for processing transmission/reception of data, respectively. Although the embodiment of the present disclosure shown in FIG. 2 is implemented in such a way that the cellular module 221, Wi-Fi module 223, BT module 225, GPS module 227, and NFC module 228 are separated from each other, an embodiment can be modified in such a way that parts of the elements (e.g., two or more) are included in an integrated chip (IC) or an IC package. For example, part of the processors corresponding to the cellular module 221, Wi-Fi module 223, BT module 225, GPS module 227, and NFC module 228, e.g., a CP corresponding to the cellular module 221 and a Wi-Fi processor corresponding to the Wi-Fi 223, may be implemented with an SoC.

The RF module 229 may transmit or receive data, e.g., RF signals. The RF module 229 may include a transceiver, a power amplifier module (PAM), a frequency filter, a low noise amplifier (LNA), and the like. The RF module 229 may also include parts for transmitting/receiving electromagnetic waves, e.g., conductors, wires, and the like, via free space during wireless communication. Although the embodiment of the present disclosure shown in FIG. 2 is implemented in such a way that the cellular module 221, Wi-Fi module 223, BT module 225, GPS module 227, and NFC module 228 share the RF module 229, an embodiment can be modified in such a way that at least one of the elements transmit or receive RF signals via a separate RF module.

The SIM card 224 may be fitted into a slot of the electronic device. The SIM card 224 may include unique identification information, e.g., integrated circuit card identifier (ICCID), or subscriber information, e.g., international mobile subscriber identity (IMSI).

The memory 230 (e.g., memory 130) includes a built-in or internal memory 232 and/or an external memory 234. The built-in memory 232 may include at least one of a volatile memory, e.g., dynamic random access memory (DRAM), static RAM (SRAM), synchronous dynamic RAM (SDRAM), and the like, a non-volatile memory, e.g., one time programmable read only memory (OTPROM), programmable ROM (PROM), erasable and programmable ROM (EPROM), electrically erasable and programmable ROM (EEPROM), mask ROM, flash ROM, NAND flash memory, NOR flash memory, and the like.

In an embodiment of the present disclosure, the built-in memory 232 may be a solid state drive (SSD). The external memory 234 may further include a flash drive, e.g., a compact flash (CF), a secure digital (SD), a micro-SD, a mini-SD, an extreme digital (XD), a memory stick, and the like. The external memory 234 may be functionally connected to the electronic device via various types of interfaces. In an embodiment of the present disclosure, the electronic device 101 may further include storage devices or storage media, such as hard drives.

The sensor module 240 may measure a physical quantity or sense operation states of the electronic device 201 and convert the measured or sensed data into electrical signals. The sensor module 240 includes at least one of a gesture sensor 240A, a gyro sensor 240B, an atmospheric pressure sensor 240C, a magnetic sensor 240D, an acceleration sensor 240E, a grip sensor 240F, a proximity sensor 240G, a color sensor 240H (e.g., red-green-blue (RGB) sensor), a biosensor 240I, a temperature/humidity sensor 240J, an luminance sensor 240K, and an ultra-violet (UV) sensor 240M.

The input system 250 includes at least one of a touch panel 252, a pen sensor 254 (i.e., a digital pen sensor), a key 256, and an ultrasonic input device 258. The touch panel 252 may sense touches using a capacitive sensing mode, a pressure sensing mode, an infrared sensing mode, and an ultrasonic sensing mode. The touch panel 252 may further include a control circuit. When the touch panel 252 is designed to operate in a capacitive sensing mode, the panel can also sense mechanical/physical touches or proximity of an object. The touch panel 252 may further include a tactile layer. In that case, the touch panel 252 can also provide tactile feedback to the user.

The pen sensor 254 (i.e., digital pen sensor) may be detected in a same or similar way as receiving a user's touch input or by using a separate recognition sheet. The key 256 may include mechanical buttons, optical keys or a key pad. The ultrasonic input device 258 is a device that can sense sounds via a microphone 288 of the electronic device 201 by using an input tool for generating ultrasonic signals, and then receiving and checking data associated with the signals. The ultrasonic input device 258 can sense signals in a wireless mode. In an embodiment of the present disclosure, the electronic device 201 may also receive a user's inputs from an external system (e.g., a computer or server) via the communication module 220.

The display module 260 (e.g., display 150) includes at least one of a panel 262, a hologram unit 264, and a projector 266. The panel 262 may be implemented with a liquid crystal display (LCD), active matrix organic light emitting diodes (AMOLEDs), or the like. The panel 262 may be implemented in a flexible, transparent, impact-resistant, and/or wearable form. The panel 262 may form a single module with the touch panel 252. The hologram unit 264 shows a three-dimensional image in the air using interference of light. The projector 266 may display images by projecting light on a screen. The screen may be placed, for example, inside or outside of the electronic device 201. In an embodiment of the present disclosure, the display module 260 may further include a control circuit for controlling the panel 262, the hologram unit 264, or the projector 266.

The interface 270 includes at least one of an HDMI 272, a USB 274, an optical interface 276, a D-subminiature (D-sub) 278, and the like. The interface 270 may also be included in the communication interface 160 shown in FIG. 1. The interface 270 may also include a mobile high-definition link (MHL) interface, an SD card, a multi-media card (MMC) interface, an infrared data association (IrDA) standard interface, or the like.

The audio module 280 may provide conversions between audio and electrical signals. At least part of the components in the audio module 280 may be included in the I/O interface 140 shown in FIG. 1. The audio module 280 may process audio output from/input to, for example, a speaker 282, a receiver 284, earphones 286, the microphone 288, and the like.

The camera module 291 may take still images or moving images. In an embodiment of the present disclosure, the camera module 291 may include one or more image sensors (e.g., on the front side and/or the back side), a lens, an image signal processor (ISP), a flash (e.g., an LED or a xenon lamp), or the like.

The power management module 295 may manage electric power supplied to the electronic device 201. The power management module 295 may include a power management integrated circuit (PMIC), a charger IC, a battery gauge, and the like.

The PMIC may be implemented in the form of an IC chip or SoC. Charging electric power may be performed in wired and/or wireless modes. The charger IC may charge a battery, and prevent input over-voltage or input over-current to the battery from a charger. In an embodiment of the present disclosure, the charger IC may be implemented with a wired charging type and/or a wireless charging type. Examples of the wireless charging type of the charger IC are a magnetic resonance type, a magnetic induction type, an electromagnetic type, an acoustic type, and the like. If the charger IC is implemented with a wireless charging type, it may also include an additional circuit for wireless charging, e.g., a coil loop, a resonance circuit, a rectifier, and the like.

The battery gauge may measure a residual amount of the battery 296, a level of voltage, a level of current, a temperature during the charge, and the like. The battery 296 stores electric power and supplies it to the electronic device 201. The battery 296 may include a rechargeable battery or a solar battery.

The indicator 297 shows states of the electronic device 201 or of the parts thereof (e.g., the AP 210), e.g., a booting state, a message state, a recharging state, and the like. The motor 298 converts an electrical signal into a mechanical vibration. Although not shown, the electronic device 201 may include a processor for supporting a mobile TV, e.g., a GPU. The mobile TV supporting processor may process media data that complies with standards of digital multimedia broadcasting (DMB), digital video broadcasting (DVB), media flow, and the like.

Each of the elements/units of the electronic device may be implemented with one or more components, and may be called different names according to the type of electronic device. The electronic device may include at least one element described above. The electronic device may also be modified in such a way as to remove part of the elements or include new elements. In addition, the electronic device may also be modified in such a way that parts of the elements are integrated into one entity that performs their original functions.

Herein, the term ‘module’ refers to a ‘unit’ including hardware, software, firmware, or a combination thereof. For example, the term ‘module’ is interchangeable with the terms ‘unit,’ ‘logic,’ ‘logical block,’ component, ‘circuit,’ and the like. A module may be the least identifiable unit or part of an integrated component. A module may also be the least unit or part thereof that can perform one or more functions of the module. A module may be implemented through mechanical or electronic modes. For example, modules according to the embodiments of the present disclosure may be implemented with at least one of an application specific integrated circuit (ASIC) chip, a field-programmable gate array (FPGAs) and a programmable-logic device that can perform functions that are known or will be developed.

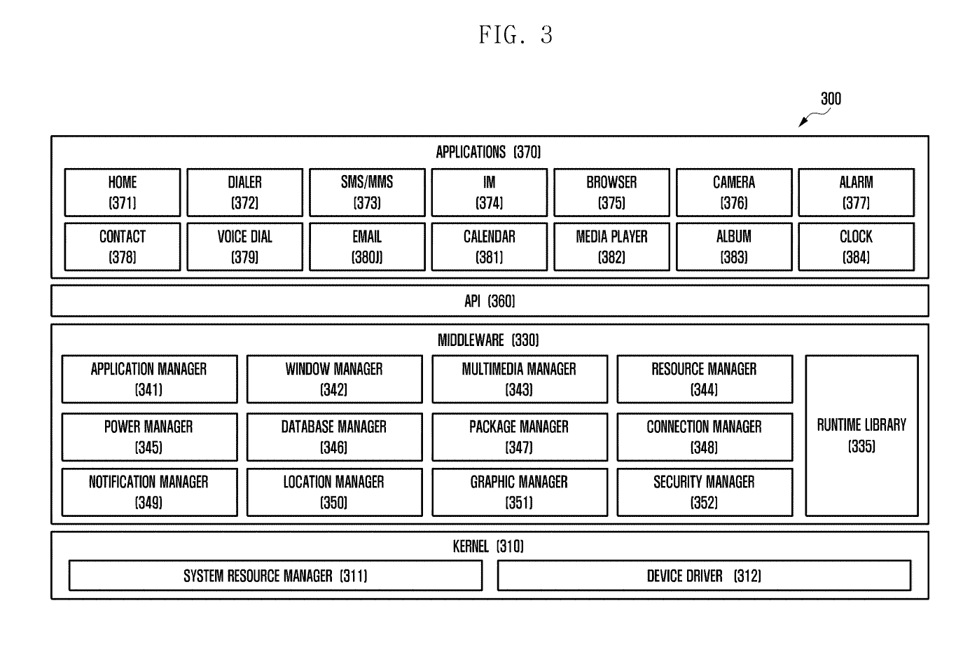

FIG. 3 is a block diagram illustrating a program module, according to an embodiment of the present disclosure.

Referring to FIG. 3, a program module 300 may include an OS for controlling resources related to the electronic device and/or various applications executed in the operating system.

The program module 300 includes a kernel 310, middleware 330, an API 360, and/or applications 370. At least some of the program module 300 may be preloaded on an electronic device, or may be downloaded from an external electronic device (e.g., the electronic device 102 or 104, or the server 106).

The kernel 310 includes, for example, a system resource manager 311 and/or a device driver 312. The system resource manager 311 may perform control, allocation, retrieval, or the like, of system resources. According to an embodiment of the present disclosure, the system resource manager 311 may include a process manager, memory manager, file system manager, or the like. The device driver 312 may include, for example, a display driver, camera driver, Bluetooth driver, shared memory driver, USB driver, keypad driver, Wi-Fi driver, audio driver, or inter-process communication (IPC) driver.

The middleware 330 may provide a function required by the applications 370 in common, or provide various functions to the applications 370 through the API 360 so that the applications 370 can efficiently use limited system resources within the electronic device. According to an embodiment of the present disclosure, the middleware 330 includes, for example, at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, and a security manager 352.

The runtime library 335 may include a library module which a compiler uses in order to add a new function through a programming language while the applications 370 are being executed. The runtime library 335 may perform input/output management, memory management, functionality for an arithmetic function, or the like.

The application manager 341 may manage, for example, a life cycle of at least one of the applications 370. The window manager 342 may manage GUI resources used for the screen. The multimedia manager 343 may determine a format required to reproduce various media files, and may encode or decode a media file by using a coder/decoder (codec) appropriate for the corresponding format. The resource manager 344 may manage resources such as a source code, memory, and storage space of at least one of the applications 370.

The power manager 345 may operate together with a basic input/output system (BIOS) to manage a battery or other power, and may provide power information required for the operation of the electronic device. The database manager 346 may generate, search for, and/or change a database to be used by at least one of the applications 370. The package manager 347 may manage the installation or update of an application distributed in the form of a package file.

The connectivity manager 348 may manage a wireless connection such as, for example, a Wi-Fi or Bluetooth connection. The notification manager 349 may display or notify of an event, such as an arrival message, an appointment, a proximity notification, and the like, in such a manner as not to disturb the user. The location manager 350 may manage location information of the electronic device. The graphic manager 351 may manage a graphic effect, which is to be provided to the user, or a user interface related to the graphic effect. The security manager 352 may provide various security functions required for system security, user authentication, and the like. According to an embodiment of the present disclosure, when the electronic device has a telephone call function, the middleware 330 may further include a telephony manager for managing a voice call function or a video call function of the electronic device.

The middleware 330 may include a middleware module that forms a combination of various functions of the above-described elements. The middleware 330 may provide a module specialized for each type of OS in order to provide a differentiated function. Also, the middleware 330 may dynamically delete some of the existing elements, or may add new elements as required.

The API 360 is, for example, a set of API programming functions, and may be provided with a different configuration according to an OS. For example, one API set may be provided for each platform, or two or more API sets may be provided for each platform.

The applications 370 include, for example, one or more applications which can provide functions such as home 371, dialer 372, SMS/MMS 373, instant message (IM) 374, browser 375, camera 376, alarm 377, contacts 378, voice dialer 379, email 380, calendar 381, media player 382, album 383, clock 384, health care (e.g., measure exercise quantity or blood sugar), and environment information (e.g., atmospheric pressure, humidity, or temperature information).

According to an embodiment of the present disclosure, the applications 370 may include an information exchange application supporting information exchange between the electronic device and an external electronic device 102 or 104. The information exchange application may include, for example, a notification relay application for transferring specific information to an external electronic device or a device management application for managing an external electronic device.

For example, the notification relay application may include a function of transferring, to the external electronic device (e.g., the first external electronic device 102 or the second external electronic 104), notification information generated from other applications of the electronic device 101 (e.g., an SMS/MMS application, e-mail application, health management application, or environmental information application). Further, the notification relay application may receive notification information from, for example, an external electronic device and provide the received notification information to a user.

The device management application may manage (e.g., install, delete, or update), for example, at least one function of an external electronic device (e.g., the electronic device 102 or 104) communicating with the electronic device (e.g., a function of turning on/off the external electronic device or some components thereof, or a function of adjusting luminance or a resolution of the display), applications operating in the external electronic device, or services provided by the external electronic device (e.g., a call service and a message service).

According to an embodiment of the present disclosure, the applications 370 may include an application (e.g., a health care application of a mobile medical device or the like) designated according to an attribute of the external electronic device (e.g., the electronic device 102 or 104). The applications 370 may include an application received from the external electronic device (e.g., the server 106, the first external electronic device 102, or the second external electronic device 104). The applications 370 may include a preloaded application or a third party application that can be downloaded from the server. Names of the elements of the program module 300 may change depending on the type of OS.

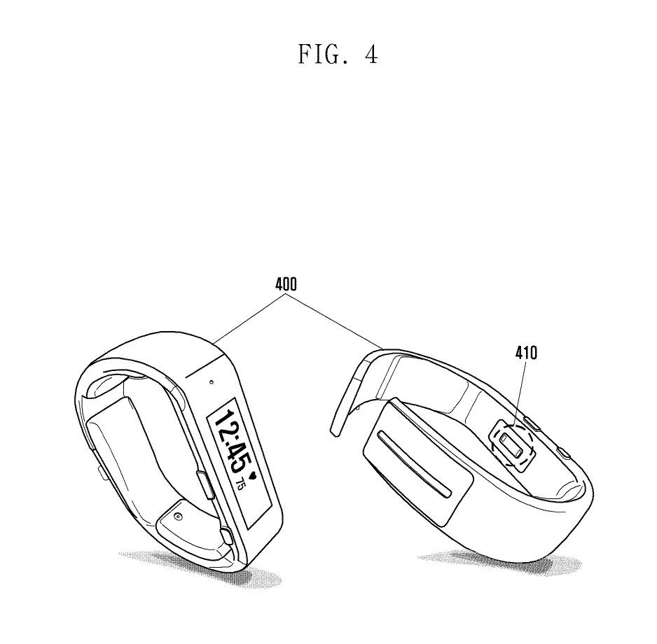

FIG. 4 is a diagram illustrating the appearance of an electronic device, according to an embodiment of the present disclosure.

In the embodiment, the electronic device may be implemented in a form that may be detachably worn on part of the user's body. The electronic device is capable of including at least one contact for contacting part of the human body, revealed on at least one of the sides. The contact (s) may also serve as a contact of a bio sensor or a contact for charging. Referring to FIG. 4, an electronic device 400 is implemented as a watch with two contacts 410 contacting the outside of the user's wrist. In another embodiment, the electronic device 400 is implemented as a watch with two contacts 410 contacting the inside of the user's wrist. Although it is not shown in FIG. 4, the electronic device 400 is capable of including a number of sensors. In this case, the electronic device 400 also includes a number of contacts according to the characteristics of the sensors.

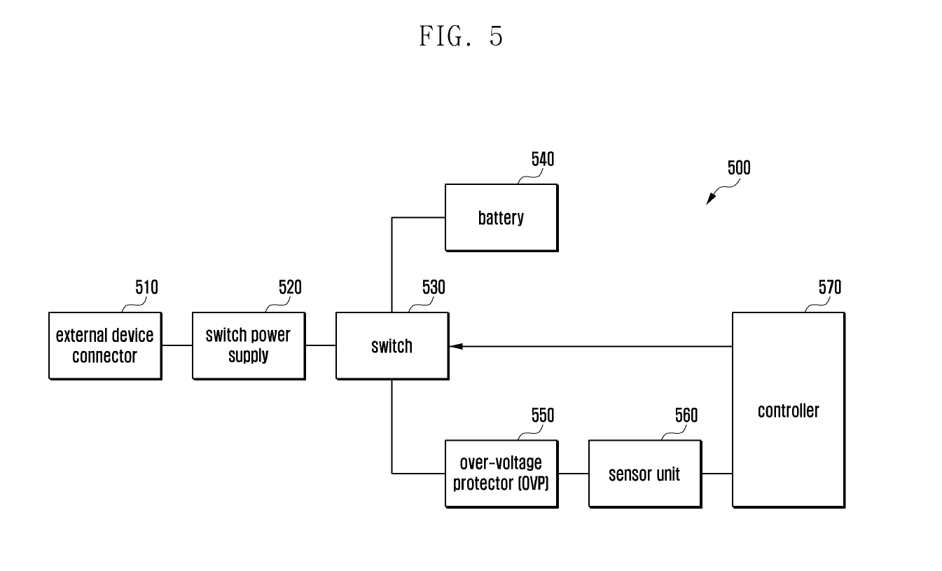

FIG. 5 is a block diagram illustrating an electronic device, according to an embodiment of the present disclosure. Referring to FIG. 5, an electronic device 500 includes at least one of an external device connector 510, a switch power supply 520, a switch 530, a battery 540, an over-voltage protector (OVP) 550, a sensor unit 560 and a controller 570.

Part or all of the functions of the individual components of the electronic device 500 may be included in at least one component shown in FIG. 1. For example, at least part of the function of the controller 570 may be included in the processor 120 shown in FIG. 1.

The external device connector 510 is electrically connected to the controller 570 and the battery 540. At least part of the external device connector 510 may be included in the input/output interface or the communication interface shown in FIG. 1. The electronic device 500 is electrically connected to an external electronic device via the external device connector 510.

The external device connector 510 is capable of including at least one contact. According to an embodiment of the present disclosure, the contacts are electrically connected to contacts of sensors of the sensor unit 560. The contacts are also electrically connected to contacts of the battery 540.

When an external electronic device includes an interface unit with an interface point, and the interface point is connected to the contact of the external device connector 510, the electronic device 500 and the external electronic device are electrically connected to each other. For example, the external electronic device may be a battery charger for charging the battery of the electronic device 500 or a storage media for storing data to/from the electronic device 500. When the electronic device 500 is electrically connected to the external electronic device via the external device connector 510, they transfer/receive data or power to/from each other.

When a user wears the electronic device 500, the contact of external device connector 510 contacts the user's skin. The sensor unit 560 is capable of sensing current flowing on the skin, via the external device connector 510. The controller 570 is capable of processing the current sensed by the sensor unit 560 to obtain a bio signal. When the battery 540 of the electronic device 500 is discharged, the switch power supply 520 is capable of: supplying power to the switch 530; controlling the switch 530 to electrically connect between the battery 540 and the external device connector 510; and electrically disconnect between the sensor unit 560 and the external device connector 510. The switch power supply 520 is capable of including at least one of the following: a gate, a voltage detector, and a low drop out (LDO) regulator. When the external device connector 510 is connected to the external electronic device, i.e., a battery charger, the switch power supply 520 electrically connects between the grounds of the battery charger and the switch 530, so that the switch 530 receives electric power from the battery charger, or the external electronic device.

According to an embodiment of the present disclosure, the switch 530, which is receiving electric power, is capable of electrically connecting between the external device connector 510 and the battery 540. The switch 530 may be set to a default, when the battery 540 is discharged, in such a way that it receives a voltage from the external electronic device and electrically connects between the external device connector 510 and the battery 540.

The switch 530 is capable of electrically connecting between the external device connector 510 and the battery 540 or between the external device connector 510 and the sensor unit 560, according to commands from the controller 570. For example, when the switch 530 receives, from the controller 570, a control signal for electrically connecting between the external device connector 510 and the sensor unit 560, it electrically connects the external device connector 510 and the sensor unit 560, and electrically disconnects the external device connector 510 and the battery 540. Similarly, when the switch 530 receives, from the controller 570, a control signal for electrically connecting between the external device connector 510 and the battery 540, it electrically connects between the external device connector 510 and the battery 540, and electrically disconnects between the external device connector 510 and the sensor unit 560.

When the switch 530 electrically connects between the external device connector 510 and the battery 540 and electrically disconnects between the external device connector 510 and the sensor unit 560, the electronic device 500 is capable of charging the battery 540 with electric power received from the external electronic device via the external device connector 510. When the switch 530 electrically connects between the external device connector 510 and a galvanic skin response (GSR) sensor of the sensor unit 560 and electrically disconnects between the external device connector 510 and the battery 540, the electronic device 500 is capable of measuring the conductivity of the human skin via the GSR sensor of the sensor unit 560.

The battery 540 supplies electric power to the electronic device 500. When the battery 540 is discharged, it is capable of performing a pre-charge process where it receives a small amount of current from the external electronic device by PMIC for a period of time. When the battery 540 is electrically connected to the external device connector 510, and the contact of the external device connector 510 is connected to the interface point of the interface of the external electronic device, the battery 540 is charged with power from the external electronic device.

The sensor unit 560 is capable of including at least one sensor, such as a GSR sensor. The GSR sensor refers to a sensor for sensing the conductivity on the human skin. The contact of the GSR sensor is selectively connected to the contact of the external device connector 510 via the switch 530. The GSR sensor is electrically connected to the external device connector 510 and measures the conductivity of the skin via the contact of the external device connector 510. For example, when the electronic device 500 is implemented as a wearable device and the user wears the electronic device 500, at least one of the contacts of the external device connector 510 contacts the user's skin, so that the GSR sensor can measure the user's skin conductivity. Information regarding the user's skin conductivity measured by the GSR sensor is transferred to the controller 570.

According to an embodiment of the present disclosure, the OVP 550 is electrically connected to the switch 530 and the sensor unit 560. The OVP 550 controls the voltage applied to the sensor unit 560 so as not to exceed a predetermined level of voltage, thereby protecting the sensor unit 560 from being damaged. For example, when the controller 570 controls the switch 530 to electrically connect the contact of the external device connector 510 to the GSR sensor, the external device connector 510 is electrically connected to the GSR sensor via the switch 530. The OVP 550 may be set to transfer a voltage of less than or equal to 1.1 V, which is permissible for the GSR sensor. When the user connects the contact of the external device connector 510 to the battery charger, i.e., the external electronic device, in order to charge the battery 540 of the electronic device 500, the electronic device 500 may receive voltage from the external device connector 510 at a level greater than 1.1 V, which is not permissible for the GSR sensor. In this case, the OVP 550 blocks the voltage from the external electronic device since the received voltage exceeds the level of voltage which is permissible for the GSR sensor.

According to an embodiment of the present disclosure, the OVP 550 is capable of transferring, to the controller 570, a signal indicating that the electronic device receives a voltage of which the level is greater than a level of voltage which is permissible for the GSR sensor. The controller 570 controls the switch 530 to electrically connect the contact of the external device connector 510 to the battery 540, so that the battery 540 can be charged with the supplied power.

The controller 570 is capable of controlling the switch 530 to electrically connect the contact of the external device connector 510 to the GSR sensor or the battery 540. When the controller 570 controls the switch 530 to electrically connect the contact of the external device connector 510 to the battery 540, the switch 530 electrically connects the external device connector 510 to the battery 540, so that the battery 540 can be charged with the voltage received via the external device connector 510.

According to an embodiment of the present disclosure, when the controller 570 controls the switch 530 to electrically connect the contact of the external device connector 510 to the sensor unit 560, the switch 530 electrically connects the external device connector 510 and the sensor unit 560, so that the sensor unit 560 performs the sensing function based on the voltage transferred via the external device connector 510. The controller 570 receives and processes information from the sensor unit 560. The controller 570 controls operations of the sensor unit 560. For example, when the sensor unit 560 includes a GSR sensor, the GSR sensor operates based on the voltage transferred via the external device connector 510 and measures the conductivity of the user's skin. The controller 570 receives and processes information related to the conductivity of the user's skin, transferred from the GSR sensor. The controller 570 controls operations of the GSR sensor.

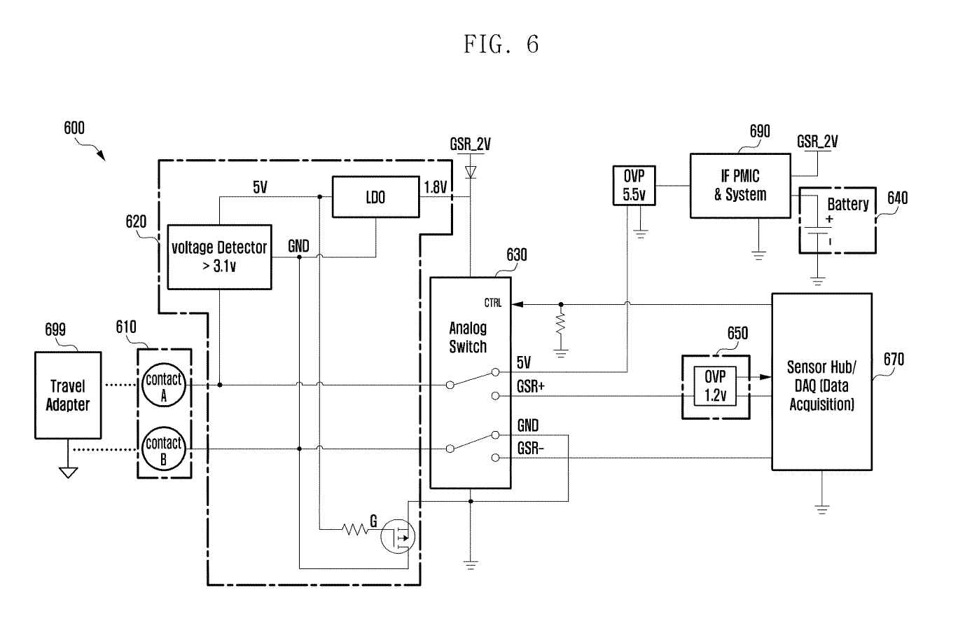

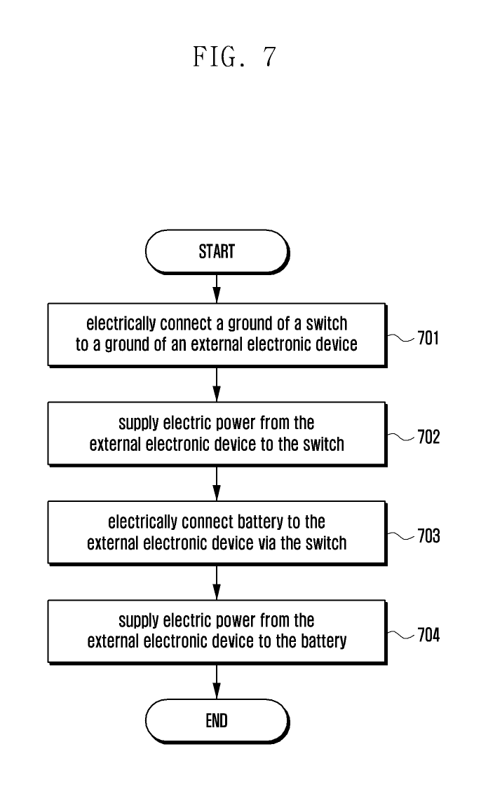

FIG. 6 is a circuit diagram illustrating an electronic device, according to an embodiment of the present disclosure, and FIG. 7 is a flow diagram illustrating operations of an electronic device, according to embodiments of the present disclosure.

Referring to FIG. 6, an electronic device 600 includes an external device connector 610 with at least one contact, a switch power supply 620, a switch 630, a battery 640, an OVP 650, and a controller 670. As shown in FIG. 6, the switch 630 is capable of electrically connecting the contact of the external device connector 610 to the battery 640 or the sensor unit 560, according to a signal from the controller 670. As shown in FIGS. 6 and 7, it is assumed that the sensor unit is a GSR sensor. The GSR sensor may be included in the controller 670. When the switch 630 electrically connects the contact of the external device connector 610 to the GSR sensor, the GSR sensor measures the conductivity of the user's skin based on the voltage transferred via the external device connector 610.

The embodiments shown in FIGS. 6 and 7 assume that the battery is discharged. The user connects the electronic device to the external electronic device. For example, the external electronic device may be a battery charger. Referring to FIG. 6, the user may connect at least one contact of the external device connector 610 to the interface point of the interface of the external electronic device (travel adapter) 699. The electronic device is capable of receiving electric power from the external electronic device 699 via at least one contact of the external device connector 610.

The electronic device electrically connects the ground of the switch to the ground of the external electronic device connected via the switch power supply 620, in operation 701.

The electronic device supplies electric power from the external electronic device to the switch, in operation 702. Referring to FIG. 6, when the battery 640 is in a discharged state, the switch 630 may be in a state where it is not electrically connected to the battery 640 or the GSR sensor. The switch 630 may receive a voltage of which the level is greater than or equal to a value, via the switch power supply 620, so that the switch 630 can electrically connect the external device connector 610 to the battery 640 or the GSR sensor.

The electronic device electrically connects the battery to the external electronic device, via the switch receiving a voltage of which the level is greater than or equal to a present value, in operation 703. Referring to FIG. 6, the switch 630 receiving a voltage electrically connects the external device connector 610 to the battery 640. According to an embodiment of the present disclosure, the switch 630 may be set to a default when the battery 640 is discharged, in such a way that it receives a voltage and electrically connects the battery 640 to the external electronic device. When the switch 630 receives a voltage, the battery 640 is connected the port 5V and GND. Therefore, the external electronic device 699 is electrically connected to the battery 640.

When the external device connector and the battery are electrically connected to each other via the switch, electric power from the external electronic device is transferred to the battery, so that the battery can be charged with the electric power, in operation 704. Referring to FIG. 6, according an embodiment of the present disclosure, it is assumed that the battery 640 is discharged. Therefore, the battery 640 cannot directly receive electric power from the battery charger. The battery 640 is capable of performing a process, e.g., a pre-charge process, where it receives a small amount of current from the external electronic device 699 under the control of the PMIC 690 connected to the battery 640 for a period of time. After performing the pre-charge process, the battery 640 is charged with electric power transferred from the external electronic device 699.

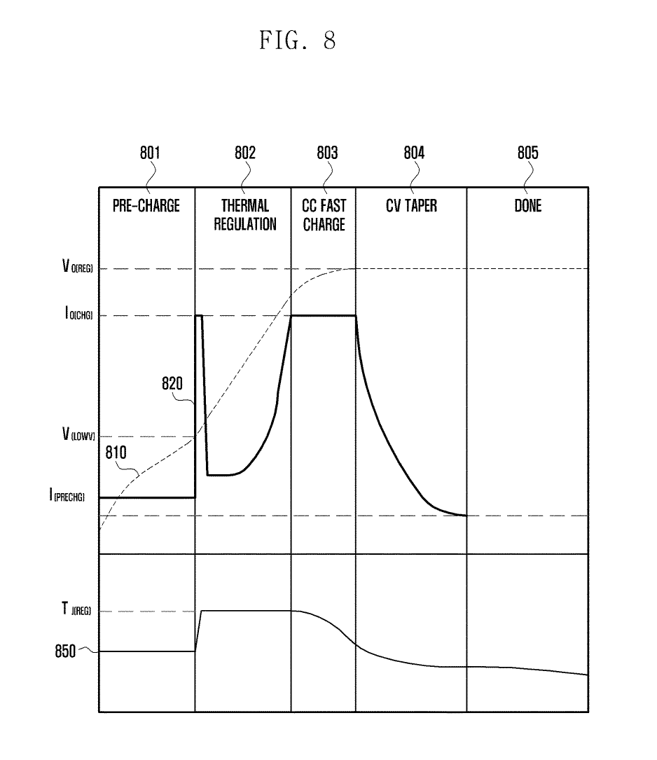

FIG. 8 is a graph showing a battery charging method, according to an embodiment of the present disclosure.

Referring to FIG. 8, the battery charging method includes five steps: pre-charge 801, thermal regulation 802, CC fast charge 803, CV Taper 804 and Done 805. Graph 810 denotes the variation of current supplied to a battery, and graph 820 denotes the voltage of a battery varying with the variation of current.

The step of the pre-charge 801 may be a process in which current, of which the amount is relatively small, e.g., I (prechg), uniformly flows from the external electronic device (e.g., a battery charger) to a battery of a voltage of which the level is less than or equal to a V (low), e.g., 2.8 V, so that the battery is charged with a voltage of up to a V (low).

The step of the thermal regulation 802 may be a process in which, when the battery is charged to have a level of voltage, a V (low), current, Io (chg), e.g., 2 A, flows from the external electronic device to the battery. When the battery receives the current of Io (chg), it may increase the level of voltage close to a Vo (reg), e.g., 5 V. When a relatively small amount of current in pre-charge 801, I (prechg), is uniformly supplied to the battery and then Io (chg) is supplied thereto in thermal regulation 802, the junction temperature of the IC may reach Tj (reg). In order to reduce the junction temperature of the IC to less than or equal to Tj (reg), the amount of current supplied to the battery is temporarily reduced and then gradually increases. Referring to FIG. 8, the junction temperature of an IC is denoted by the graph 850.

The step of CC fast charge 803 may be a process in which, when the battery becomes close to a level of voltage, Vo (reg), the current, lo (chg), flows into the battery for a certain period of time (for example, until the battery reaches a Vo (reg)), without increasing the current. The junction temperature of an IC may be lowered as the amount of current is temporarily decreased.

The step of CV Taper 804 and Done 805 may be processes in which, the battery reaches a level of voltage, a Vo (reg), and retains the Vo (reg), the amount of current supplied to the battery may decrease. When current no long flows into the battery, the process of charging the battery may be interrupted.

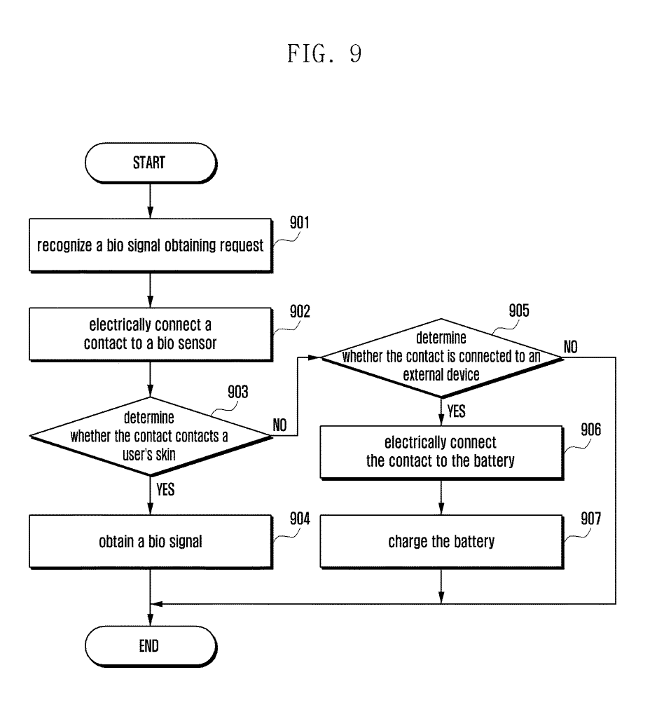

FIG. 9 is a flow diagram illustrating operations of an electronic device, according to an embodiment of the present disclosure.

The embodiment of FIG. 9 is described, based on a case in which the battery has not been discharged, e.g., a case where the electronic device is still operable, using electric power from the battery.

Referring to FIG. 9, the controller of the electronic device transfers a command for obtaining a bio signal to a switch, in operation 901. The switch is capable of recognizing a bio signal obtaining request signal from the controller. Referring to FIG. 6, the controller 670 (sensor hub/data acquisition (DAQ)) may transfer a control signal for obtaining a bio signal, e.g., a user's skin conductivity, to the GSR sensor. For example, the controller 670 may transfer a high level signal to the switch 630

The electronic device connects the contact of the external device connector to the bio sensor, in operation 902. Referring to FIG. 6, the switch 630 receives the high level signal from the controller 670 and is connected to ports GSR+ and GSR−, thereby electrically connecting the contact of the external device connector 610 to the GSR sensor.

The electronic device determines whether the contact contacts the user's skin, in operation 903. When the electronic device ascertains that the contact contacts the user's skin, it obtains a bio signal from the bio sensor, in operation 904. Referring to FIG. 6, when at least one contact of the external device connector 610 contacts the user's skin, the controller 670 may receive information related to a user's measured skin conductivity from the contact of the external device connector electrically connected to the GSR sensor.

When the electronic device ascertains that the contact does not contact the user's skin, it determines whether the contact contacts the external electronic device, in operation 905. When the electronic device ascertains that the contact contacts the external electronic device in operation 905, it electrically connects the contact of the external device connector to the battery, in operation 906.

Referring to FIG. 6, the controller 670 is capable of transferring, to the switch 630, a control signal for electrically connecting to the GSR sensor. At least one of the contacts of the external device connector 610 may be connected not to a user's skin but to an interface point of the interface of the external electronic device 699. In this case, since the contact of the external device connector 610 is electrically connected to the GSR sensor via the switch 630, a high level of voltage from the external electronic device 699 may be transferred to the GRS sensor via the OVP 650. The GSR sensor and other bio sensors may be damaged when receiving a voltage of which the level is greater than or equal to a certain value. In order to prevent the sensors from being damaged, an over-voltage protector (OVP) 650 is employed. The OVP 650 is capable of transferring, to the controller 670, a signal indicating that it received a high level of voltage from the switch 630. After receiving the signal, the controller 670 is capable of transferring a control signal for electrically connecting to the battery 640. The switch 630 receives the control signal from the controller 670 and electrically connects the contact of the external device connector 610 to the battery 640.

The electronic device transfers electric power, received via the contact of the external device connector, to the battery, thereby charging the battery, in operation 907. For example, referring to FIG. 6, the battery 640 may be charged with electric power from the external electronic device 699.

According to an embodiment of the present disclosure, the OVP 650 may block the voltage from the switch 630 to protect the GSR sensor from being damaged.

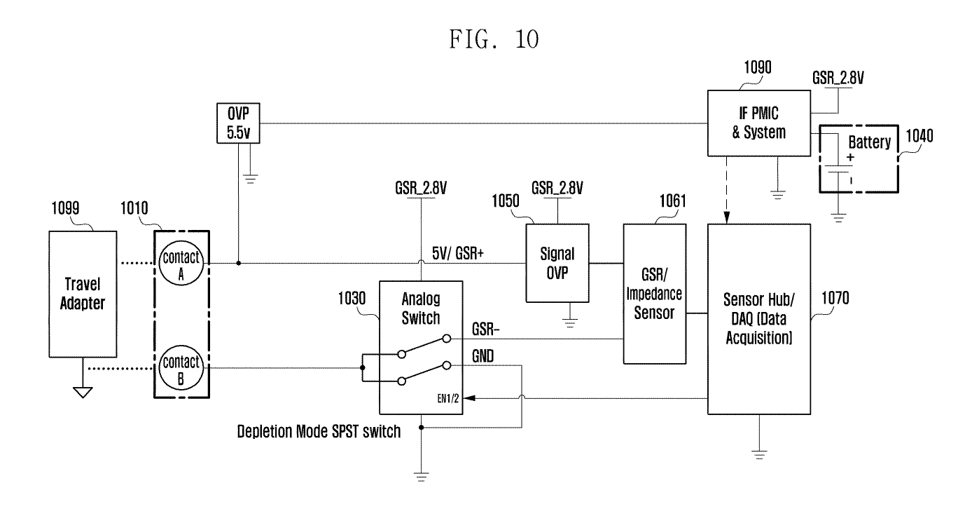

FIG. 10 is a circuit diagram illustrating an electronic device, according to an embodiment of the present disclosure.

Referring to FIG. 10, the electronic device includes an external device connector 1010 with at least one contact, a switch 1030, a battery 1040, an OVP 1050 and a controller 1070. The GSR sensor may be included in the controller 1070.

As shown in FIG. 10, the switch 1030 is capable of electrically connecting the contact of the external device connector 1010 to the battery 1040 or the GSR sensor. When the switch 1030 electrically connects the contact of the external device connector 1010 to the GSR sensor, the GSR sensor is capable of measuring a user's skin conductivity, based on its received voltage.

According to an embodiment of the present disclosure, the switch 1030 may not need a switch power supply. In the embodiment shown in FIG. 10, the switch 1030 may be a depletion mode switch. Although the battery 1040 is discharged, the switch 1030 may be connected to both GSR− and GND ports. When the battery 1040 is discharged and the switch 1030 is in the state described above, the user may connect at least one contact of the external device connector 1010 to an interface point of the external electronic device 1099. The electronic device may receive electric power from the external electronic device (travel adapter) 1099 via at least one contact of the external device connector 1010.

When the battery 1040 is discharged, the battery 1040 may not immediately be charged with the same electric power as it is received. The battery 1040 is capable of performing a pre-charge process where it receives a small amount of current from the external electronic device 1099 under the control of the PMIC 1090 connected to the battery 1040 for a period of time. After the battery 1040 performs the pre-charge process, the external device connector 1010 receives a voltage for charging the battery 1040, e.g., 5 V, from the external electronic device 1099. The external device connector 1010 transfers the received voltage of 5 V to the switch 1030, and the switch 1030 is connected to the GSR− and GND ports, according to the properties of the switch 1030. Therefore, the switch 1030 electrically connects the contact of the external device connector 1010 to the GSR sensor or the battery 1040.

Accordingly, the switch 1030 transfers the received voltage of 5 V to the battery 1040 or the GSR sensor based on a control signal. The GSR sensor and other bio sensors may be damaged when receiving a voltage of which the level is greater than or equal to a certain value. In order to prevent the sensors from being damaged, an OVP 1050 is employed. The OVP 1050 may block the voltage transferred via the external device connector 1010 to protect the GSR sensor from being damaged. For example, referring to FIG. 10, when the OVP 1050 receives a voltage greater than or equal to 2.8 V via the external device connector 1010, it may block the voltage. As the contact of the external device connector 1010 is connected to the battery 1040, the voltage of 5 V is transferred to the battery 1040, so that it can be charged with 5 V.

According to an embodiment of the present disclosure, when the battery 1040 has not been discharged, the switch 1030 may receive at least two signals (e.g., first and second signals) from the controller 1070 (sensor hub/data acquisition (DAQ)).

TABLE 1

| Vcc | EN1 | EN2 | Switch 1 | Switch 2 |
|---|---|---|---|---|
| LOW | X | X | ON | ON |
| HIGH | HIGH | HIGH | OFF | OFF |
| HIGH | LOW | HIGH | ON | OFF |
| HIGH | HIGH | LOW | OFF | ON |

Table 1 is a truth table for the control of a depletion mode switch. Referring to Table 1, when the controller 1070 transfers a low level of signal as a first signal (EN1) and a high level of signal as a second signal (EN2) to the switch 1030, the switch 1030 may electrically connect the contact of the external device connector 1010 to the GSR sensor. When at least one of the contacts of the external device connector 1010 contacts a user's skin, the controller 1070 may receive information related to the user's skin conductivity from the external device connector.

According to an embodiment of the present disclosure, when the controller 1070 transfers a high level of signal as a first signal and a low level of signal as a second signal to the switch 1030, the switch 1030 may electrically connect the contact of the external device connector 1010 to the battery 1040. Since at least one of the contacts of the external device connector 1010 is connected to an interface point of the interface of the external electronic device 1099, the electric power is transferred from the external electronic device 1099 to the battery 1040, so that the battery 1040 can be charged with the received power.

When the controller 1070 transfers first and second signals in high level to the switch 1030, the switch 1030 does not connect the external device connector 1010 to the battery 1040 or the GSR sensor.

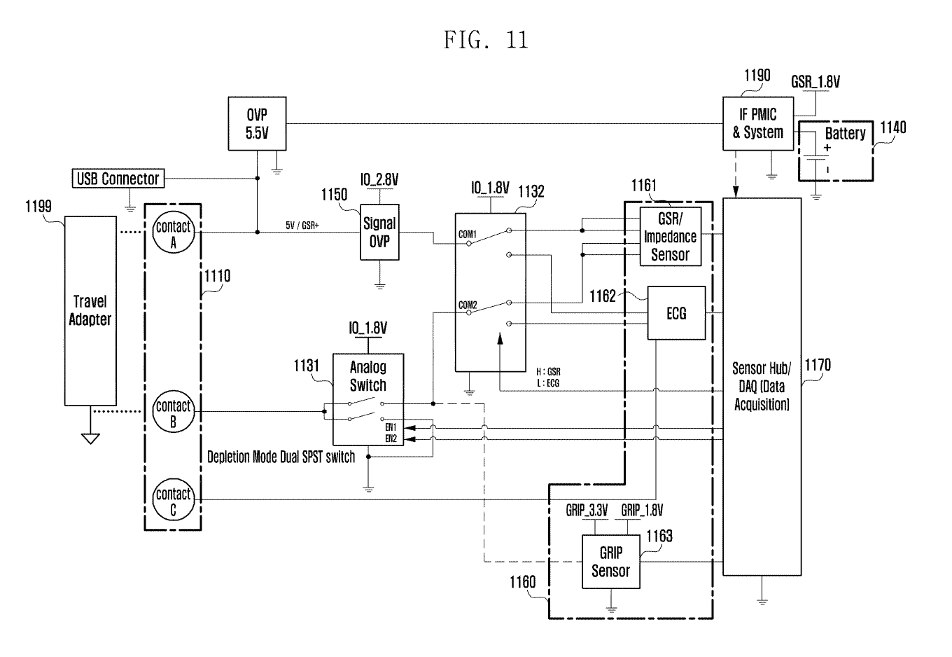

FIG. 11 is a circuit diagram illustrating an electronic device, according to an embodiment of the present disclosure. Referring to FIG. 11, the electronic device includes an external device connector 1110 with two or more contacts, two or more switches 1131 and 1132, a battery 1140, an OVP 1150, a sensor unit 1160 with a number of sensors, and a controller 1170.

According to an embodiment of the present disclosure, the sensor unit 1160 of the electronic device is capable of including a number of sensors. The external device connector 1110 is capable of including two or more contacts for a number of sensors and the battery 1140, and also a number of contacts according to properties of sensors. The electronic device may also include a number of switches for electrically connecting the contact of the external device connector 1110 to a number of sensors and the battery 1140.

Referring to FIG. 11, the sensor unit 1160 includes a GSR/impedance sensor 1161, an electrocardiography (ECG) sensor 1162, and a grip sensor 1163. The external device connector 1110 may include at least one contact. An example of the sensor included in the electronic device is an ECG sensor with three contacts. The electronic device includes a first switch 1131 and a second switch 1132. The first switch 1131 is capable of electrically connecting the contact of the external device connector 1110 to the sensor unit 1160 or the battery 1140. The second switch 1132 is capable of electrically connecting the contact of the external device connector 1110 to the GSR sensor 1161 or the ECG sensor 1162. The grip sensor 1163 may be electrically connected to the output terminal of the first switch 1131 or an antenna and perform the sensing operation.

According to an embodiment of the present disclosure, the first switch 1131 and/or the second switch 1132 may not need a switch power supply according to the features. The first switch 1131 and/or the second switch 1132 may be a depletion mode switch. For example, when the first switch 1131 is a depletion mode switch, although the battery 1140 is discharged, the first switch 1131 may be connected to the second switch 1132 and the GND port, without the switch power supply.

According to an embodiment of the present disclosure, when the battery 1140 is discharged and the first switch 1131 is connected to the second switch 1132 and the GND port, the user may connect at least one contact of the external device connector 1110 to an interface point of the external electronic device (travel adapter) 1199. The battery 1140 of the electronic device may receive electric power from the external electronic device 1199 via at least one contact of the external device connector 1110.

When the battery 1140 is discharged, the battery 1140 may not immediately be charged with the same electric power as it is received due to its properties. The battery 1140 is capable of performing a pre-charge process where it receives a small amount of current from the external electronic device 1199 under the control of the PMIC 1190 connected to the battery 1140 for a period of time. After the battery 1140 performs the pre-charge process, the external device connector 1110 receives a voltage for charging the battery 1140, e.g., 5 V, from the external electronic device 1199.

The external device connector 1110 transfers the received voltage of 5 V to the second switch 1132. Since the first switch 1131 is connected to the second switch 1132 and the GND port, the contact of the external device connector 1110 may be electrically connected to the sensor unit 1160 and the battery 1140.

Therefore, the received voltage of 5 V is transferred to the battery 1140 and the sensor unit 1160. The sensors may be damaged when receiving a voltage of which the level is greater than or equal to a certain value. In order to prevent the sensors from being damaged, an OVP 1150 is employed. The OVP 1150 may block the voltage transferred via the external device connector 1110 to protect the sensors from being damaged. For example, referring to FIG. 11, when the OVP 1150 receives a voltage greater than or equal to 2.8 V via the external device connector 1110, it may block the voltage. In a state where the external device connector 1110 and the sensors are electrically disconnected from each other, the external device connector 1110 is electrically connected to the battery 1140, and thus the voltage of 5 V is transferred to the battery 1140, so that it can be charged with 5 V.

According to an embodiment of the present disclosure, when the battery 1140 has not been discharged, the controller 1070 transfers at least two signals (e.g., first and second signals) to the first switch 1131. The controller 1170 may transfer at least one signal to the second switch 1132.

Referring to Table 1, when the controller 1170 transfers a high level of signal as a first signal and a low level of signal as a second signal to the first switch 1131, the first switch 1131 may electrically connect the external device connector 1110 to the battery 1140. Since at least one of the contacts of the external device connector 1110 is connected to an interface point of the interface of the external electronic device 1199, the battery 1140 is charged with electric power transferred from the external electronic device 1199.

When the controller 1170 transfers a low level signal as a first signal and a high level signal as a second signal to the first switch 1131, and a high level signal to the second switch 1132, the first switch 1131 electrically connects the contact of the external device connector 1110 to the sensors. The high level signal that the controller 1170 transferred to the second switch 1132 may be a command for electrically connecting the external device connector 1110 and the GSR sensor (impedance sensor) 1161. The second switch 1132 receives the high level of signal from the controller 1170, and electrically connects the external device connector 1110 to the GSR sensor 1161. When at least one of the contacts of the external device connector 1110 contacts the user's skin, the controller 1170 is capable of obtaining information related to the user's skin conductivity via the GSR sensor 1161.

When the controller 1170 transfers a low level signal as a first signal and a high level signal as a second signal to the first switch 1131, and a low level signal to the second switch 1132, the first switch 1131 electrically connects the contact of the external device connector 1110 to the sensors. The low level signal that the controller 1170 transferred to the second switch 1132 may be a command for electrically connecting the external device connector 1110 and an electrocardiogram (ECG) sensor. The second switch 1132 receives the low level signal from the controller 1170 and electrically connects the external device connector 1110 to the ECG sensor. When at least three contacts of the external device connector 1110 contact a user's skin, the controller 1170 is capable of obtaining information related to an electrocardiogram via the ECG sensor.

For example, since a grip sensor as one of the sensors is electrically connected to the first switch 1131, it may measure a degree of grip without a command that the first switch 1131 may receive from the controller 1170, and transfer the measured grip-related information to the controller 1170.

According to an embodiment of the present disclosure, when the controller 1170 transfers first and second signals in high level to the first switch 1131, the first switch 1131 does not connect the external device connector 1110 to the battery 1140 or the sensor unit 1160.

According to various embodiments of the present disclosure, the wearable electronic device includes a housing; a battery built in the housing; a coupling member connected to part of the housing, for detachably coupling the electronic device to part of the user's body; a conductive member which is revealed outside from one side of the housing or one side of the coupling member and is electrically connected to the battery; at least one sensor electrically connected to the conductive member; and a circuit electrically connected to the battery, the conductive member, and the at least one sensor. The conductive member comprises one or more contacts. The circuit monitors a voltage and an amount of current received via the one or more contacts, and charges the battery with the current or operates the at least one sensor, based on part of the monitored values.

According to various embodiments of the present disclosure, if the battery is discharged, the circuit selectively charges the battery with the current or operates the at least one sensor, based on electric power from an external device electrically connected to the conductive member.

According to various embodiments of the present disclosure, the one side of the housing or the one side of the coupling member contacts part of a user's body when the user wears the electronic device.

According to various embodiments of the present disclosure, the conductive member includes an antenna.

According to various embodiments of the present disclosure, the conductive member includes part of the coupling member.

According to various embodiments of the present disclosure, the circuit detects a resistance of an external object connected to the conductive member by using at least one sensor.

According to various embodiments of the present disclosure, the circuit further comprises controller configured to control a switch to obtain a signal from the at least one sensor or charge the battery via the one or more contacts and the switch electrically connecting the one or more contacts to at least one sensor and the battery.

According to various embodiments of the present disclosure, wherein, if a signal obtaining command is received from the controller, the circuit electrically connects the one or more contacts to the at least one sensor, receives current from an external object, and obtains the signal from the external object based on the received current.

According to various embodiments of the present disclosure, wherein, if a level of voltage related to the current transferred from the external object is greater than a preset value, the controller is further configured to control the switch to electrically connect at least one of the one or more contacts to the battery and charge the battery with the current from the external object. The preset value is a level of voltage permissible for the bio sensor.

According to various embodiments of the present disclosure, a level of voltage related to the current from the external object is transferred to an over-voltage protector (OVP); and the controller is further configured to receive, from the OVP, a signal indicating that the level of voltage is greater than the preset value, and control the switch to electrically connect at least one of the one or more contacts to the battery, according to the received signal.

According to various embodiments of the present disclosure, wherein, if a battery charging command is received from the controller, the controller is further configured to control the switch to electrically connect at least one of the one or more contacts to the battery, so that the battery is charged with the current transferred from the external object.

According to various embodiments of the present disclosure, the method of obtaining a bio signal by a wearable electronic device includes monitoring a voltage and an amount of current received via the one or more contacts, and charging the battery with the current or operating at least one bio sensor, based on at least one of the voltage and the amount of current.

According to various embodiments of the present disclosure, operating the at least one bio sensor includes connecting the one or more contacts to the at least one bio sensor, receiving current from an external object, and obtaining a bio signal from the external object based on the received current.

According to various embodiments of the present disclosure, operating the at least one bio sensor includes if a bio signal obtaining request from the at least one bio sensor is recognized, electrically connecting the one or more contacts to the at least one bio sensor; and obtaining a bio signal via the at least one bio sensor.

According to various embodiments of the present disclosure, charging the battery with the current includes if a level of voltage related to the current transferred from the external object is greater than a preset value, controlling the switch to electrically connect at least one of the one or more contacts to the battery and charging the battery with the current from the external object, wherein the preset value is a level of voltage permissible for the at least one bio sensor.

According to various embodiments of the present disclosure, charging the battery with the current includes if a battery charging request is recognized, controlling a switch to electrically connect the one or more contacts to a battery, according to the battery charging request; and charging the battery with the current from the external object.

According to various embodiments of the present disclosure, charging the battery with the current includes if a battery of the electronic device is fully discharged, controlling the switch to electrically connect one or more contacts to the battery; and charging the battery with the current transferred from the external object.

According to various embodiments of the present disclosure, charging the battery includes performing a pre-charge process to transfer the current, having an amount that is less than or equal to a preset value, from the external object, to the battery for a certain period of time.

According to various embodiments of the present disclosure, a non-transitory computer-readable recording medium having recorded thereon a program configured to monitor a voltage and an amount of current received via the one or more contacts, and charge the battery with the current or operating at least one bio sensor, based on at least one of the voltage and the amount of current.

As described above, the bio signal obtaining method and electronic device is capable of precisely measuring the temperature on the skin via an external device connector (e.g., POGO pin, etc.), without using a structure which is additionally installed to the electronic device (e.g., a wearable device) and senses a bio signal. An example of the structure is a contact configured to contact the human skin. Since the electronic device is simplified in terms of structure, it reduces the manufacturing cost and is advantageous in terms of design or part installation, etc.

The bio signal obtaining method is advantageous because it can be easily applied to wearable devices which are decreasing in size.

The bio signal obtaining method and electronic device is capable of measuring reliable bio signals, applying the bio signals to various application fields, and providing various and precise services by cooperating with other sensors installed to the electronic device.

While the disclosure has been shown and described with reference to certain embodiments thereof, it will be understood by those skilled in the art that various changes in form and detail may be made therein without departing from the spirit and scope of the disclosure as defined by the appended claims.

What is claimed is:

1. A wearable electronic device comprising:
a housing;
a battery disposed in the housing;
a coupling member, connected to the housing, for detachably coupling the electronic device to a part of a user's body;
a conductive member exposed on a side of the housing or on a side of the coupling member and electrically connected to the battery, the conductive member comprising one or more contacts;
at least one sensor electrically connected to the conductive member; and
a circuit electrically connected to the battery, the conductive member, and the at least one sensor,
wherein the circuit monitors a voltage and an amount of current received via the one or more contacts, and charges the battery with the current or operates the at least one sensor, based on at least one of the voltage and the amount of current.

2. The wearable electronic device of claim 1, wherein, if the battery is fully discharged, the circuit selectively charges the battery with the current or operates the at least one sensor, based on electric power from an external device electrically connected to the conductive member.

3. The wearable electronic device of claim 1, wherein the side of the housing or the side of the coupling member contacts the part of the user's body when the user wears the electronic device.

4. The wearable electronic device of claim 1, wherein the conductive member comprises an antenna.

5. The wearable electronic device of claim 1, wherein the conductive member comprises part of the coupling member.

6. The wearable electronic device of claim 1, wherein the circuit detects a resistance of an external object connected to the conductive member using the at least one sensor.

7. The wearable electronic device of claim 1, wherein the circuit further comprises:
a controller configured to control a switch to obtain a signal from the at least one sensor or charge the battery via the one or more contacts; and
the switch electrically connecting the one or more contacts to at least one sensor and the battery.

8. The wearable electronic device of claim 7, wherein, if a signal obtaining command is received from the controller, the circuit electrically connects the one or more contacts to the at least one sensor, receives current from an external object, and obtains the signal from the external object based on the received current.

9. The wearable electronic device of claim 7, wherein, if a level of voltage related to the current transferred from the external object is greater than a preset value, the controller is further configured to control the switch to electrically connect at least one of the one or more contacts to the battery and charge the battery with the current from the external object, and
wherein the preset value is a level of voltage permissible for the bio sensor.

10. The wearable electronic device of claim 9, wherein:
a level of voltage related to the current from the external object is transferred to an over-voltage protector (OVP); and
the controller is further configured to receive, from the OVP, a signal indicating that the level of voltage is greater than the preset value, and control the switch to electrically connect at least one of the one or more contacts to the battery, according to the received signal.

11. The wearable electronic device of claim 7, wherein, if a battery charging command is received from the controller, the controller is further configured to control the switch to electrically connect at least one of the one or more contacts to the battery, so that the battery is charged with the current transferred from the external object.

12. A method of obtaining a bio signal by a wearable electronic device, the method comprising:
monitoring a voltage and an amount of current received via the one or more contacts, and
charging the battery with the current or operating at least one bio sensor, based on at least one of the voltage and the amount of current.

13. The method of claim 12, wherein operating the at least one bio sensor comprises:
connecting the one or more contacts to the at least one bio sensor;
receiving current from an external object; and
obtaining a bio signal from the external object based on the received current.

14. The method of claim 12, wherein operating the at least one bio sensor comprises:
if a bio signal obtaining request from the at least one bio sensor is recognized, electrically connecting the one or more contacts to the at least one bio sensor; and
obtaining a bio signal via the at least one bio sensor.

15. The method of claim 12, wherein charging the battery with the current comprises:
if a level of voltage related to the current transferred from the external object is greater than a preset value, controlling the switch to electrically connect at least one of the one or more contacts to the battery; and
charging the battery with the current from the external object,
wherein the preset value is a level of voltage permissible for the at least one bio sensor.

16. The method of claim 12, wherein charging the battery with the current comprises:
if a battery charging request is recognized, controlling a switch to electrically connect the one or more contacts to a battery, according to the battery charging request; and
charging the battery with the current from the external object.

17. The method of claim 13, wherein charging the battery with the current comprises:
if a battery of the electronic device is fully discharged, controlling the switch to electrically connect one or more contacts to the battery; and
charging the battery with the current transferred from the external object.

18. The method of claim 17, wherein charging the battery comprises:
performing a pre-charge process to transfer the current, having an amount that is less than or equal to a preset value, from the external object, to the battery for a certain period of time.

19. A non-transitory computer-readable recording medium having recorded thereon a program configured to perform a method comprising:
monitoring a voltage and an amount of current received via the one or more contacts; and
charging the battery with the current or operating at least one bio sensor, based on at least one of the voltage and the amount of current.
